(19) United States
(12) Patent Application Publication
(10) Pub. No.: US 2008/0020471 A1
(43) Pub. Date: Jan. 24, 2008

(54) Blood sugar detecting system

(75) Inventors: Chi-Min Chau, Dadu Township (TW); Chih-Hsien Su, Kaohsiung City (TW); Hsiang-Yuan Huang, Kaohsiung City (TW); Yuh-Jiuan Lin, Taishan Township (TW)

Correspondence Address: BIRCH STEWART KOLASCH & BIRCH, PO BOX 747, FALLS CHURCH, VA, 22040-0747, US

(73) Assignee: INDUSTRIAL TECHNOLOGY RESEARCH INSTITUTE

(21) Appl. No.: 11/703,190

(22) Filed: Feb. 7, 2007

(30) Foreign Application Priority Data
Jul. 18, 2006 (TW) 95126283

(51) Int. Cl. G01N 33/49

(52) U.S. Cl. 436/63

(57) ABSTRACT

A blood sugar detecting system using emission quantum dots is provided. A non-cyclodextran carbohydrate-containing molecule and a glucose-recognizing molecule respectively bind to an emission quantum dot and a light-absorbing molecule to form the blood sugar measuring system. When the non-cyclodextran carbohydrate-containing molecule and the glucose-recognizing molecule bind together to bring the emission quantum dot very close to the light-absorbing molecule, a fluorescence resonance energy transfer effect is happened between them. Glucose can compete with the non-cyclodextran carbohydrate-containing molecule for the binding site of the glucose-recognizing molecule to detect glucose concentration variation.

### RELATED APPLICATIONS

The present application is based on, and claims priority from, Taiwan Application Serial Number 95126283, filed Jul. 18, 2006, the disclosure of which is hereby incorporated by reference herein in its entirety.

### BACKGROUND

1. Field of Invention

The present invention relates to a blood sugar detecting system. More particularly, the present invention relates to a blood sugar detecting system using nanomaterial.

2. Description of Related Art

At present, the number of diabetics is up to 1.4 billions in the world and increased in a rate of 1% per year. The mean age of the diabetics is decreasing. Therefore, the number of the diabetics is estimated to be 2.3 billions in 2020. Accordingly, the blood sugar (glucose) becomes an important index to health. Monitoring the blood sugar level can not only avoid the problems brought by the high blood sugar level, but also prevent the problems brought by low blood sugar level.

The blood sugar monitoring method can be simply classified into blood plasma sugar examination and whole blood sugar examination. In plasma sugar examination, blood is drawn and separated into plasma and blood cells. The plasma is then examined to determine glucose concentration in plasma. In the whole blood sugar examination, a small needle is used to stab a finger to get a drop of whole blood. After dropping the blood on a test paper, a small blood sugar meter is used to determine the glucose concentration in the blood. In the methods described above, the glucose detecting reagents can be used only one time; therefore the cost is quite considerable in long time blood sugar monitoring.

### SUMMARY

A detecting system of blood sugar using emission quantum dot is provided. A first detecting system includes emission quantum dot—glucose-recognizing molecule and non-cyclodextran carbohydrate-containing molecule—light-absorbing molecule. A second detecting system includes emission quantum dot—non-cyclodextran carbohydrate-containing molecule and glucose-recognizing molecule—light-absorbing molecule.

The emission quantum dot emits light in a range from ultraviolet to infrared after being excited. The absorption spectrum of the light-absorbing molecule at least partially overlaps the emission spectrum of the emission quantum dot.

When the non-cyclodextran binds to the glucose-recognizing molecule, the distance between the emission quantum dot and the light-absorbing molecule is very close and fluorescence resonance energy transfer (FRET) effect will be generated. Glucose in a blood sample and the non-cyclodextran carbohydrate-containing molecule compete with the binding site of the glucose-recognizing molecule.

According to an embodiment of this invention, the emission quantum dot comprises a quantum dot of II-VI semiconductor or III-V semiconductor. The quantum dot of II-VI semiconductor is CdSe quantum dot, CdTe quantum dot or CdSe—ZnS core-shell quantum dot. The quantum dot of III-V semiconductor is InP quantum dot, GaN quantum dot or InAs-core/GaAs-shell quantum dot.

According to another embodiment of this invention, the glucose-recognizing molecule comprises a carbohydrate binding ligand, such as lectin. The lectin comprises Concanavalin A (Con A), peanut agglutinin (PNA), wheat germ agglutinin (WGA) or soybean agglutinin (SBA).

According to another embodiment of this invention, the light-absorbing molecule comprises a quantum dot of III-V semiconductor or II-VI semiconductor. The quantum dot of III-V semiconductor is InP quantum dot, GaN quantum dot or InAs-core/GaAs-shell quantum dot. The quantum dot of II-VI semiconductor is CdS quantum dot, CdSe quantum dot, CdTe quantum dot, ZnS quantum dot, ZnSe quantum dot, or ZnTe quantum dot.

According to another embodiment of this invention, the light-absorbing molecule comprises Au nanoparticles.

According to another embodiment of this invention, the light-absorbing molecule comprises organic fluorochrome, inorganic fluorochrome, or organic quencher.

According to another embodiment of this invention, the non-cyclodextran carbohydrate-containing molecule comprises a glycoprotein, a glycolipid, or a carbohydrate. The carbohydrate is a monosaccharide, a disaccharide, an oligosaccharide, a polysaccharide or derivatives thereof. The monosaccharide is, for example, glucose or mannose. The disaccharide is, for example, maltose, lactose, or sucrose. The polysaccharide is, for example, starch, cellulose, or dextran. The derivative of monosaccharide is, for example, sialic acid.

As embodied and broadly described herein, a reusable blood sugar detecting reagent is provided to reduce the cost of consuming materials.

It is to be understood that both the foregoing general description and the following detailed description are made by use of examples and are intended to provide further explanation of the invention as claimed.

### BRIEF DESCRIPTION OF THE DRAWINGS

The invention can be more fully understood by reading the following 1 detailed description of the preferred embodiment, with reference made to the accompanying drawings as follows:

FIG. 1 is a diagram of a blood sugar detecting system according to an embodiment of this invention;

FIG. 2 is a diagram showing a process to prepare the blood sugar detecting system according to an embodiment of this invention; and

FIG. 3 is a fluorescence spectrum showing the intensity variation before and after the glucose and dextran-TMR compete with the binding site of CdSe—ZnS quantum dot—Con A.

### DETAILED DESCRIPTION

### I. Detecting System of Blood Sugar

A blood sugar detecting system using emission quantum dot is provided. The detecting system comprises emission quantum dot (QD), glucose-recognizing molecule, non-cyclodextran carbohydrate-containing (NCCC) molecule, and light-absorbing molecule.

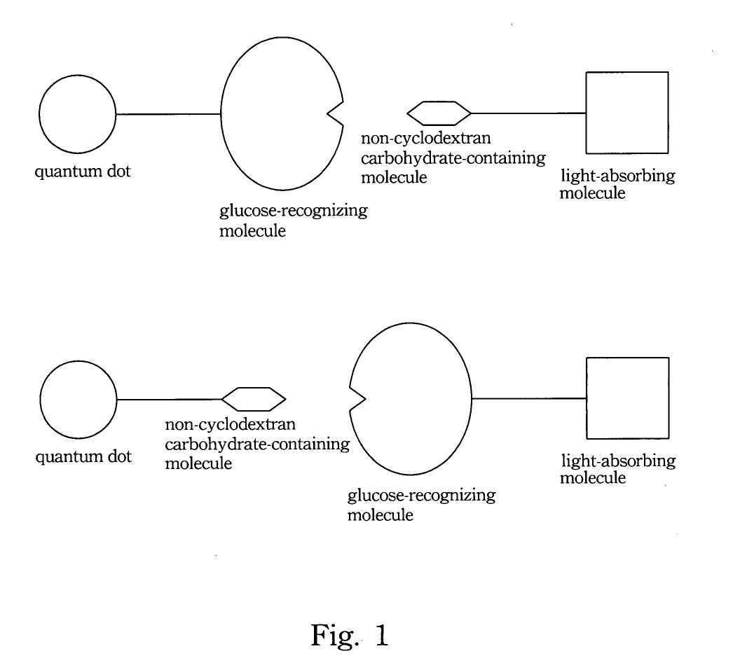

FIG. 1 is a diagram of a blood sugar detecting system according to an embodiment of this invention. In FIG. 1, two combinations are shown. In the first combination, the emission QD and the glucose-recognizing molecule are conjugated together, and the NCCC molecule and the light-absorbing molecule are conjugated together. In the second combination, the emission QD and the NCCC molecule are conjugated together, and the glucose-recognizing molecule and the light-absorbing molecule are conjugated together.

The light-absorbing molecule can be, for example, a fluorochrome or quencher. When the NCCC molecule binds at the binding site of the glucose-recognizing molecule, the distance between the emission quantum dot and the light-absorbing molecule is less than 50-100 Å to allow fluorescence resonance energy transfer (FRET) to occur between them. Hence, the absorption spectrum of the light-absorbing molecule has to at least partially overlap the emission spectrum of the emission quantum dot. It allows the light-absorbing molecule to absorb the emitted light of the emission QD to emit light of longer wavelength (in the case of fluorochrome) or simply absorb the light without any emission (in the case of quencher).

When glucose is presented in the environment of the detecting system described above, the glucose will compete the binding site of the glucose-recognizing molecule with the NCCC molecule. The FRET is stopped after the glucose binds at the binding site of the NCCC molecule, since the competition occurs and then distance between the emission QD and the light-absorbing molecule is increased. Therefore, the glucose concentration in the environment can be determined by detecting the emission strength variation of the emission QD or the fluorochrome.

The emission QD comprises a quantum dot of II-VI semiconductor or III-V semiconductor. The quantum dot of II-VI semiconductor is CdSe quantum dot, CdTe quantum dot or CdSe—ZnS core-shell (CdSe—ZnS) quantum dot. The quantum dot of III-V semiconductor is InP quantum dot, GaN quantum dot or InAs-core/GaAs-shell (InAs—GaAs) quantum dot.

When the emission QD is in the excited state, light is emitted in the range from ultraviolet to Infrared. Researchers are trying to apply semiconductor QDs to light emitting biosensors, since the photostability of the semiconductor QDs is batter than conventional dyes and the emission spectrum is continually tunable by adjusting the size of the semiconductor QDs (CdSe—ZnS Quantum Dots as Resonance Energy Transfer Donors in a Model Protein—Protein Binding Assay, Nano Lett., 2001, vol. 1, p. 469; Self-Assembled Nanoscale Biosensors Based on Quantum Dot FRET Donors, Nature Materials, 2003, Vol. 2, p. 630). Therefore, the size of the semiconductor QDs can be adjusted and determined by the absorption spectrum of the light-absorbing molecule to overlap the emission spectrum of the QDs and the absorption spectrum of the light-absorbing molecule to facilitate the FRET.

The glucose-recognizing molecule described above comprises a carbohydrate binding ligand, such as lectin. The lectin comprises Concanavalin A (Con A), peanut agglutinin (PNA), wheat germ agglutinin (WGA) or soybean agglutinin (SBA). These molecules are common glucose-recognizing molecules. For example, Con A is disclosed to be a glucose-recognizing molecule in U.S. Pat. No. 6,844,166 incorporated herein by reference entirely. Con A can recognize a-linkage mannose and glucose located on the end of polysaccharide chain.

The non-cyclodextran carbohydrate-containing molecule described above comprises a glycoprotein, a glycolipid, or a carbohydrate. The carbohydrate is a monosaccharide, a disaccharide, an oligosaccharide, a polysaccharide or derivatives thereof. The monosaccharide is, for example, glucose or mannose. The disaccharide is, for example, maltose, lactose, or sucrose. The polysaccharide is, for example, starch, cellulose, or dextran. The derivative of monosaccharide is, for example, sialic acid.

Many of the fluorochromes described above are commercially available. For example, many fluorochromes are listed in column 10, lines 19-34 of U.S. Pat. No. 6,844,166, which is entirely incorporated herein by reference, and thus omitted here. Therefore, a suitable fluorochrome depends on the emission spectrum of the emission QD can be chosen.

Many of the quenchers described above are also commercially available, and a suitable quencher depends on the emission spectrum of the emission QD can be thus chosen. For example, the quencher includes Au nanoparticles (Inhibition Assay of Bimolecules based on Fluorescence Resonance Energy Transfer (FRET) between Quantum Dots and Gold Nanoparticles, J. Am. Chem. Soc. 2005, Vol. 127, p.3270), III-V semiconductor QDs, II-VI semiconductor QDs, or some of the commercial molecules shown below in Formula I-IV. The III-V semiconductor QDs described can be, for example, InP quantum dot, GaN quantum dot or InAs-core/GaAs-shell quantum dot. The II-VI semiconductor QDs described above can be, for example, CdS quantum dot, CdSe quantum dot, CdTe quantum dot, ZnS quantum dot, ZnSe quantum dot, or ZnTe quantum dot.

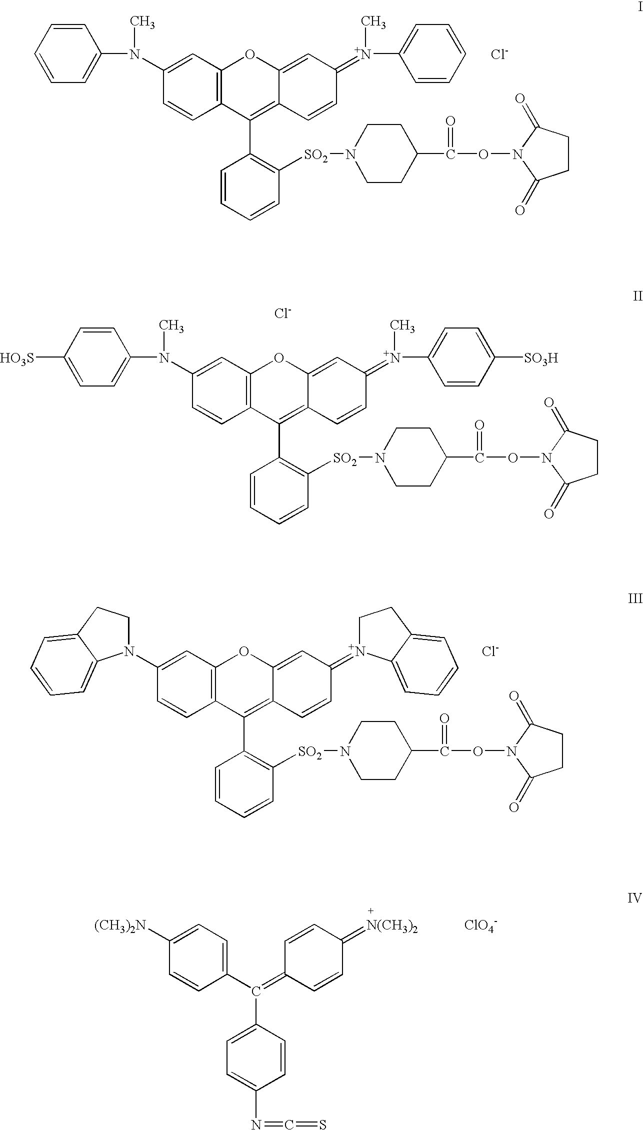

### II. Method of Preparing the Detecting System of Blood Sugar

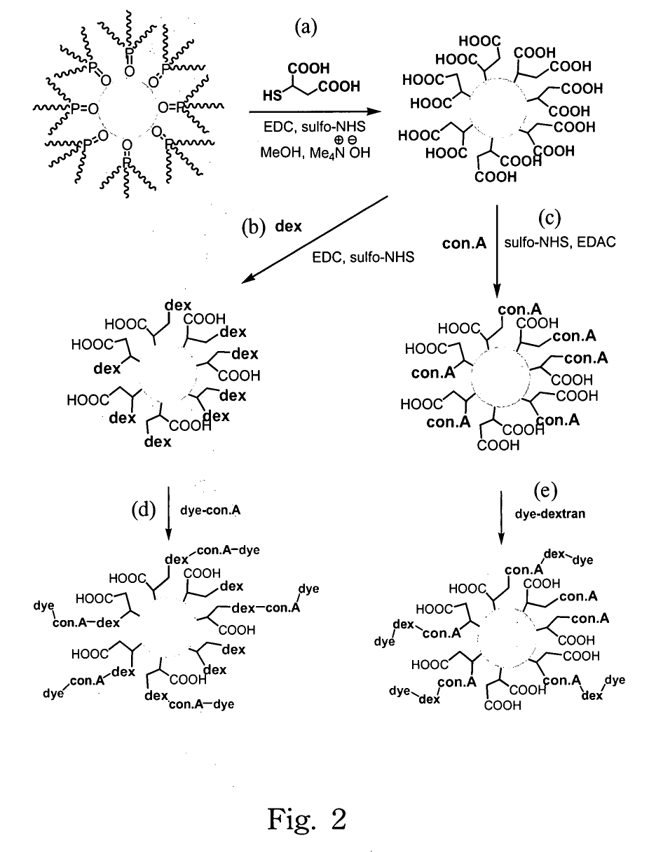

FIG. 2 is a diagram showing a process to prepare the blood sugar detecting system according to an embodiment of this invention. Some real examples are described below to further illustrate the blood sugar detecting system using emission QD as described above.

### Preparation Method of CdSe—ZnS QDs Coated with MSA

The preparation methods of CdSe—ZnS QD and surface modification thereof to allow biomolecules to attach on the surface of CdSe—ZnS QD had been studied in some literatures. For example, some are reviewed in Quantum Dots in Biology and Medicine (Physica E, 2004, Vol. 25, p. 1). The surface of CdSe—ZnS QD was modified by mercapto-succinic acid (MSA) in this embodiment.

First, CdSe—ZnS QD was synthesized. A Cd precursor was dissolved in PO(n-Oct)₃ (tri-n-octylphosphine oxide; TOPO), and a Se precursor was dissolved in P(n-Oct)₃ (tri-n-octylphosphine; TOP). The Cd solution and the Se solution were mixed under a dry ambient without oxygen and stirred at 320° C.
The temperature was then decreased to 290° C. to crystallize CdSe. After the desired crystal size was obtained, a TOP solution containing Zn and S was slowly dropped into the mixed solution described above to have ZnS coated on the CdSe QD. After stop adding the TOP solution containing Zn and S, the temperature was decreased again to stop reaction and then CdSe—ZnS QD coated with TOPO can be obtained.

In step (a) of FIG. 2, the CdSe—ZnS QD coated with TOPO (8.2 mg) was dispersed in methanol (10 mL). After being vibrated by a supersonic vibrator for 30 min, MSA/MeOH (50 mg/5 mL) was added to the methanol solution of CdSe—ZnS QDs coated with TOPO. The pH of the methanol solution was then adjusted to about 10 by adding tetramethyl-ammonium hydroxide (3 M). The reaction solution was refluxed for 8 hrs. After cooling, ethanol (20 mL) was added and then centrifugated at 10,000 rpm. The process described above was repeated for 3 times. Finally, the solution containing CdSe—ZnS QDs coated with MSA was filtered through 0.22 μm filter to obtain the CdSe—ZnS QDs coated with MSA.

### Preparation Method of CdSe—ZnS—Dextran Complex

In step (b) of FIG. 2, CdSe—ZnS coated with MSA (1.54 mg/mL), 1-(3-dimethylaminopropyl)-3-ethylcarbodiimide (EDC, 7.7 mg/mL), N-Hydroxysulfo-succinimide (sulfo-NHS, 0.3 mg/mL), and amino functionalized dextran (4 mg/mL) were mixed in phosphate buffer solution (1 mL, PBS, pH 7.4) to form a mixed solution. The mixed solution was reacted at room temperature for 24 hrs. Then, the mixture was centrifugated under a gravity of 5,000 g for 30 min. The process described above was repeated for 3 times to remove excess reactants. The obtained precipitations were CdSe—ZnS—dextran complex.

### Preparation Method of CdSe—ZnS—Con A Complex

In Step (c) of FIG. 2, CdSe—ZnS coated with MSA (1.54 mg/mL), EDC (7.7 mg/mL) and sulfo-NHS (0.3 mg/mL) and Con A (3 mg/mL) were mixed in 1 mL PBS to react at 4° C. for 24 hrs. Next, the mixture was centrifugated under a gravity of 3,000 g for 40 min. The process described above was repeated for 5 times to remove excess reactants. The obtained precipitations were CdSe—ZnS—Con A complex.

### Dextran—Light-Absorbing Molecule Complex

The dextran—light-absorbing molecule complex was purchased from Molecular Probes, Catalog No. D1816: dextran, tetramethylrhodamine (TMR) conjugate.

### Con A—Light-Absorbing Molecule Complex

The Con A—light-absorbing molecule complex was purchased from Molecular Probes, Catalog No. C-860: concanavalin A, tetramethylrhodamine (TMR) conjugate.

### Test of Recognition Between Dextran and Con A

Recognition test between CdSe—ZnS—ConA complex and dextran—TMR complex or between CdSe—ZnS—dextran complex and Con A—TMR complex was performed to know whether the step (d) or step (e) in FIG. 2 was probable. CdSe—ZnS (emission at 596 nm, red light)—Con A complex (1.5 mg/mL) reacted with CdSe—ZnS (emission at 568 nm, green light)—dextran complex (1.5 mg/mL) for 3 min. Under ultraviolet radiation, a mixture of green light and red light could be observed by eyes. In addition, precipitations of CdSe—ZnS (red light)—Con A complex/CdSe—ZnS (green light)—dextran complex were clearly observed.

Accordingly, after being modified by CdSe—ZnS, Con A and dextran could still recognize each other. The protein structure of Con A was not affected by CdSe—ZnS conjugation. Hence, step (d) or step (e) in FIG. 2 was probable.

### Competition Reaction Between Glucose and Dextran

Dextran (molecular weight 10K, 5 mg/mL) was added into the solution of CdSe—ZnS (red light)—Con A complex (2 mg/mL) to aggregate the CdSe—ZnS (red light)—Con A complexes to form precipitations. Glucose was then added to react for 3 min. During the reaction period, glucose competed the binding sites of Con A with dextran, and the precipitations disappeared. This proofed that glucose can compete the binding sites of Con A with dextran.

### FRET Effect Between CdSe—ZnS—Con A Complex and Dextran—Light-Absorbing Molecule Complex

The product, CdSe—ZnS (emission at 571 nm)—Con A complex (0.5-1.5 mg/mL) and dextran—TMR (emission at 580 nm) complex (0.5 mg/mL), of step (e) in FIG. 2 was chosen to be the test system for detecting glucose. The CdSe—ZnS (emission at 571 nm)—Con A complex was used as an energy donor, and the dextran—TMR (emission at 580 nm) complex was used as an energy acceptor.

When the solutions of two complexes described above were mixed, the fluorescence at 571 nm was decreased. The results are shown in the table below. Hence, the fluorescence energy of CdSe—ZnS QD was successfully transferred to TMR. That is, FRET was occurred between CdSe—ZnS QD and TMR.

| CdSe—ZnS (emission at 571 nm) - | Decreased intensity of |
|---|---|
| Con A complex (mg/mL) | emission at 571 nm (%) |
| 0.5 | 6.0 |
| 1.0 | 9.3 |
| 1.5 | 15.0 |

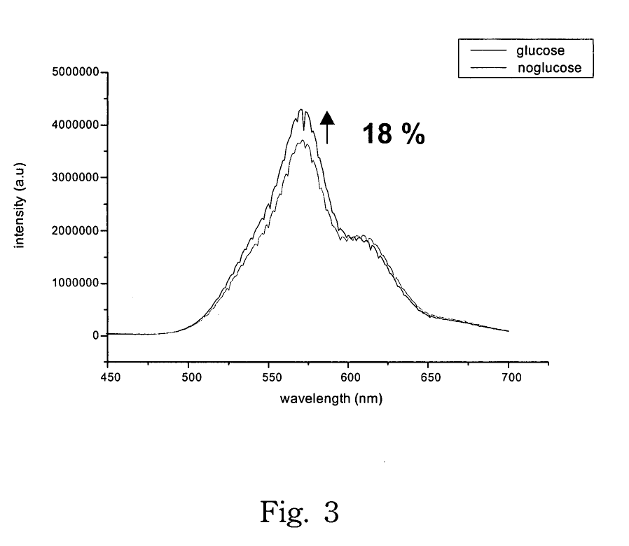

Referring to FIG. 3, glucose (300 mg/mL) were then added into the mixture solution, the fluorescent intensity of CdSe—ZnS QD was increased by about 18%. Hence, glucose can dissociate the CdSe—ZnS (emission at 571 nm)—Con A complex to decrease the FRET effect between CdSe—ZnS QD and TMR.

Accordingly, a glucose detecting system of emission QD—glucose-recognition molecule/dextran—light-absorbing molecule or emission QD—dextran/glucose-recognition molecule—light-absorbing molecule can be used to detect glucose concentration variation in the environment. In addition, since the competition reaction between glucose and the dextran to the binding site of the glucose-recognition molecule is reversible, the detecting system can be repeatedly used. Thus, the detecting system is suitable for monitoring blood sugar concentration variation in long term to prevent patient fingers from being stabbed by needles.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A blood sugar detecting system, comprising:
an emission quantum dot having a emission spectrum in a range from ultraviolet light to infrared light;
a glucose-recognizing molecule;
a light-absorbing molecule having a absorption spectrum, which at least partially overlaps the emission spectrum of the emission quantum dot; and
a non-cyclodextran carbohydrate-containing molecule,
the non-cyclodextran carbohydrate-containing molecule binding the surface of the emission quantum dot when the glucose-recognizing molecule binds the light-absorbing molecule,
the non-cyclodextran carbohydrate-containing molecule binding the light-absorbing molecule when the glucose-recognizing molecule binds the surface of the emission quantum dot.

2. The detecting system of claim 1, wherein the emission quantum dot comprises a quantum dot of II-VI semiconductor or III-V semiconductor.

3. The detecting system of claim 2, wherein the quantum dot of II-VI semiconductor is CdSe quantum dot, CdTe quantum dot or CdSe—ZnS core-shell quantum dot.

4. The detecting system of claim 2, wherein the quantum dot of III-V semiconductor is InP quantum dot, GaN quantum dot or InAs-core/GaAs-shell quantum dot.

5. The detecting system of claim 1, wherein the glucose-recognizing molecule comprises a carbohydrate binding ligand.

6. The detecting system of claim 5, wherein the carbohydrate binding ligand comprises a lectin.

7. The detecting system of claim 6, wherein the lectin comprises Concanavalin A (Con A), peanut agglutinin (PNA), wheat germ agglutinin (WGA) or soybean agglutinin (SBA).

8. The detecting system of claim 6, wherein the lectin comprises Concanavalin A (Con A).

9. The detecting system of claim 1, the light-absorbing molecule comprises a quantum dot of III-V semiconductor.

10. The detecting system of claim 9, wherein the quantum dot of III-V semiconductor is InP quantum dot, GaN quantum dot or InAs-core/GaAs-shell quantum dot.

11. The detecting system of claim 1, wherein the light-absorbing molecule comprises a quantum dot of II-VI semiconductor.

12. The detecting system of claim 11, wherein the quantum dot of II-VI semiconductor is CdS quantum dot, CdSe quantum dot, CdTe quantum dot, ZnS quantum dot, ZnSe quantum dot, or ZnTe quantum dot.

13. The detecting system of claim 1, wherein the light-absorbing molecule comprises Au nanoparticles.

14. The detecting system of claim 1, wherein the light-absorbing molecule comprises organic fluorochrome, inorganic fluorochrome, or organic quencher.

15. The detecting system of claim 1, wherein the non-cyclodextran carbohydrate-containing molecule comprises a glycoprotein, a glycolipid, or a saccharide

16. The detecting system of claim 15, wherein the saccharide is a monosaccharide, a disaccharide, an oligosaccharide, a polysaccharide, or derivatives thereof.

17. The detecting system of claim 16, wherein the monosaccharide is glucose, mannose.

18. The detecting system of claim 16, wherein the disaccharide is maltose, lactose, or sucrose.

19. The detecting system of claim 16, wherein the polysaccharide is starch, cellulose, or dextran.

20. The detecting system of claim 16, wherein the polysaccharide is dextran.

21. The detecting system of claim 16, wherein the derivative of monosaccharide is sialic acid.
